(19) United States
(12) Patent Application Publication
(10) Pub. No.: US 2011/0130591 A1
(43) Pub. Date: Jun. 2, 2011

(54) STEROSELECTIVE SYNTHESIS OF CERTAIN TRIFLUOROMETHYL-SUBSTITUTED ALCOHOLS

(75) Inventors: Daniel R. FANDRICK, Danbury, CT (US); Jonathan T. REEVES, New Milford, CT (US); Jinhua J. SONG, Hopewell Junction, NY (US)

(73) Assignee: BOEHRINGER INGELHEIM INTERNATIONAL GMBH, Ingelheim am Rhein (DE)

(21) Appl. No.: 12/788,560

(22) Filed: May 27, 2010

Related U.S. Application Data
(60) Provisional application No. 61/183,610, filed on Jun. 3, 2009.

(51) Int. Cl. C07C 231/12

(52) U.S. Cl. 564/176

(57) ABSTRACT

A process for synthesis of a compound of Formula (X)

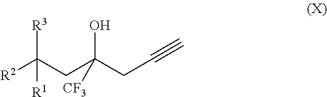

wherein:
R¹ is an aryl group substituted with one to three substituent groups,
   wherein each substituent group of R¹ is independently C₁-C₅ alkyl, aminocarbonyl, alkylaminocarbonyl, dialkylaminocarbonyl, halogen, carboxy, cyano, or trifluoromethyl,
      wherein each substituent group of R¹ is optionally independently substituted with one to three substituents selected from C₁-C₃ alkyl, C₁-C₃ alkoxy, phenyl, and alkoxyphenyl; and
R² and R³ are each independently C₁-C₅ alkyl.

### FIELD OF THE INVENTION

The present invention relates to the stereoselective synthesis of certain trifluoromethyl-substituted alcohols.

### BACKGROUND OF THE INVENTION

Trifluoromethyl-substituted alcohols of Formula (I) have been described as ligands that bind to the glucocorticoid receptor. These compounds are potential therapeutics in treating a number of diseases modulated by glucocorticoid receptor function, including inflammatory, autoimmune and allergic disorders. Examples of these compounds are described in U.S. Pat. Nos. 7,268,152; 7,189,758; 7,186,864; 7,074,806; 6,960,581; 6,903,215; and 6,858,627, which are each incorporated herein by reference in their entireties and are hereinafter termed “the Trifluoromethyl-Substituted Alcohol Patent Applications”.

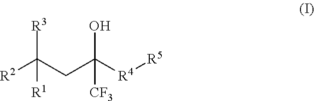

It is well known in the art that enantiomers of a particular compound can have different biological properties including efficacy, toxicity, and pharmacokinetic properties. Thus, it is often desirable to administer one enantiomer of a racemic therapeutic compound.

The synthetic methods disclosed in the patent applications cited above describe the synthesis of racemic products. Separation of enantiomers was accomplished by chiral HPLC and may be accomplished by other conventional ways of separating enantiomers. Chiral HPLC and other enantiomer separation method, however, are generally unsuitable for large-scale preparation of a single enantiomer. Thus, a stereoselective synthesis for preparation of these compounds would be highly desirable.

The present invention discloses a synthesis of certain compounds of Formula (X)

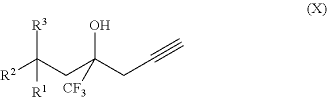

which are key intermediates in the synthesis of enantiomerically pure compounds of Formula (I).

### SUMMARY OF THE INVENTION

The instant invention is directed to a process for synthesis of a compound of Formula (X)

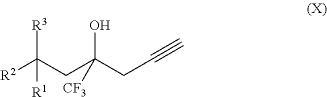

wherein:
R¹ is an aryl group substituted with one to three substituent groups,
wherein each substituent group of R¹ is independently C₁-C₅ alkyl, aminocarbonyl, alkylaminocarbonyl, dialkylaminocarbonyl, halogen, carboxy, cyano, or trifluoromethyl,
   wherein each substituent group of R¹ is optionally independently substituted with one to three substituents selected from C₁-C₃ alkyl, C₁-C₃ alkoxy, phenyl, and alkoxyphenyl; and
      R² and R³ are each independently C₁-C₅ alkyl;
      the process comprising:
(a) reacting a dioxaborolane of Formula (A) with a trialkylsilyl alkyne of Formula (B), in a suitable solvent, in the presence of a suitable base with or without a metal halide, such as magnesium chloride, and subsequently adding acetyl chloride to provide an alkynyl borolane of Formula (C)

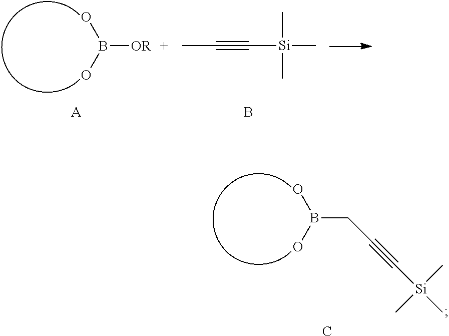

(b) reacting the alkynyl borolane of Formula (C) with a suitable trifluoromethyl ketone of Formula (D), in the presence of a organometallic complex generated from the reaction of dialkyl zinc and a suitable N-alkyl-L-proline, in a suitable solvent, at a suitable temperature, and subsequently adding a suitable acid, such as phosphoric acid, to the reaction mixture to form a mixture of trimethylsilyl alkynes of Formula (E) and (E′)

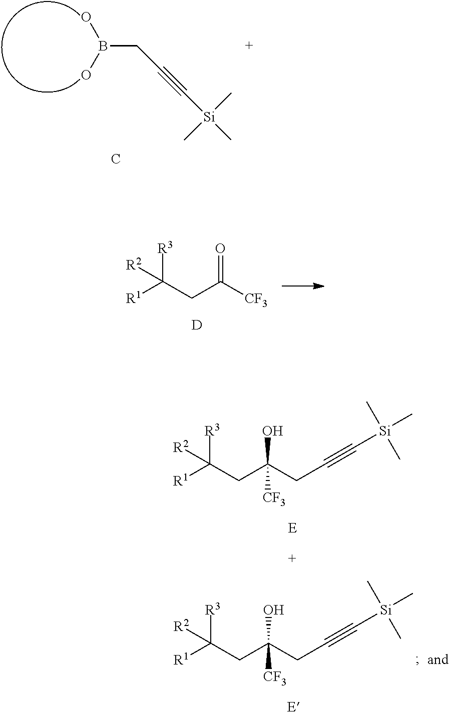

(c) reacting the trimethylsilyl alkyne of Formula (E) or (E′) with a suitable base, such as sodium hydroxide or an alkoxide base, at a suitable temperature, to provide a compound of Formula (X) or (X′) respectively

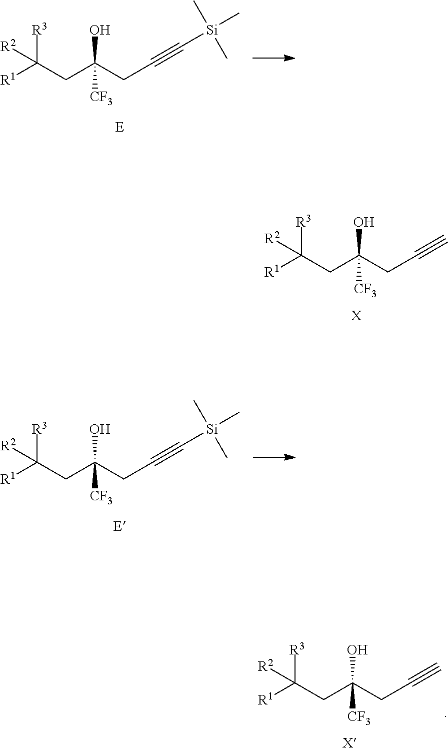

The compound of Formula (X) or (X′) may be converted to another compound of Formula (X) or (X′) by reactions known to one skilled in the art.

Another aspect of the invention includes the above process for the synthesis of a compound of Formula (X), wherein:

R¹ is an aryl group substituted with one to three substituent groups,
wherein each substituent group of R¹ is independently C₁-C₅ alkyl, aminocarbonyl, alkylaminocarbonyl, halogen, carboxy, cyano, or trifluoromethyl,
   wherein each substituent group of R¹ is optionally independently substituted with one to three substituents selected from C₁-C₃ alkyl, phenyl, and alkoxyphenyl; and
      R² and R³ are each independently C₁-C₃ alkyl.

In an aspect of the invention, the dioxaborolane of step (a) is 2-methoxy-4,4,5,5-tetramethyl-1,3,2-dioxaborolane or 2-isopropoxy-4,4,5,5-tetramethyl-1,3,2-dioxaborolane.

In an aspect of the invention, the trialkylsilane alkyne of step (a) is 1-triethylsilyl-1-propyne, 1-trimethylsilyl-1-propyne, 1-triisopropylsilyl-1-propyne, 1-(t-butyl-dimethylsilyl)-1-propyne, or 1-(tert-butyldiphenylsilyl)-1-propyne, preferably 1-trimethylsilyl-1-propyne.

In an aspect of the invention, the suitable solvent of step (a) is diethyl ether, dipropyl ether, diisopropyl ether, dibutyl ether, tetrahydrofuran (THF), ethylene glycol dimethyl ether (DME), tert-butyl methyl ether (MTBE), or a mixture thereof, preferably diethyl ether or THF.

In an aspect of the invention, the suitable base for step (a) is n-butyl lithium, sec-butyl lithium, tert-butyl lithium, or n-pentyl lithium, preferably n-butyl lithium.

In an aspect of the invention, the suitable metal halide for step (a) is magnesium chloride, magnesium bromide, or magnesium triflate, preferably magnesium chloride.

In an aspect of the invention, the trifluoromethyl ketone compound (D) for step (b) is 5-fluoro-N-(4-methoxybenzyl)-2-(4,4,4-trifluoro-1,1-dimethyl-3-oxobutyl)benzamide, 4-(5-bromo-2-methoxyphenyl)-1,1,1-trifluoro-4-methylpentan-2-one, or 5-fluoro-N-[(5)-1-(4-methoxyphenyl)ethyl]-2-(4,4,4-trifluoro-1,1-dimethyl-3-oxobutyl)benzamide.

In an aspect of the invention, the suitable aqueous acid of step (b) is hydrochloride acid, hydrobromic acid, sulfuric acid, trifluoroacetic acid, acetic acid, phosphoric acid, or ammonium chloride, preferable aqueous hydrochloric acid

In an aspect of the invention, the suitable dialkyl zinc of step (b) is dimethyl zinc, diethyl zinc, or diisopropyl zinc, preferable diethyl zinc.

In an aspect of the invention, the suitable N-alkyl-L-proline of step (b) is N-methyl-L-proline, N-ethyl-L-proline, N-isobutyl-L-proline, N-isopropyl-L-proline, N-cyclopentyl-L-proline, N-cyclohexyl-L-proline, N-tert-butyl-L-proline, or N-3-pentyl-L-proline, preferably N-isopropyl-L-proline or N-cyclopentyl-L-proline.

In an aspect of the invention, the suitable temperature of step (b) is from −78° C. to 30° C.

In an aspect of the invention, the suitable base of step (c) is sodium hydroxide, potassium hydroxide, cesium hydroxide, sodium methoxide, sodium ethoxide, sodium isopropoxide, or sodium tert-butoxide, preferable sodium methoxide.

In another aspect of the invention, the suitable temperature of step (c) is 0° C. to 50° C.

It should be noted that the invention should be understood to include none, some, or all of these various aspects in various combination.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### A. Chemical Nomenclature, Terms, and Conventions

In the groups, radicals, or moieties defined below, the number of carbon atoms is often specified preceding the group, for example, C₁-C₁₀ alkyl means an alkyl group or radical having 1 to 10 carbon atoms. The term “lower” applied to any carbon-containing group means a group containing from 1 to 8 carbon atoms, as appropriate to the group (i.e., a cyclic group must have at least 3 atoms to constitute a ring). In general, for groups comprising two or more subgroups, the last named group is the radical attachment point, for example, “alkylaryl” means a monovalent radical of the formula Alk-Ar—, while “arylalkyl” means a monovalent radical of the formula Ar-Alk- (where Alk is an alkyl group and Ar is an aryl group). Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

The terms “alkyl” or “alkyl group” mean a branched or straight-chain saturated aliphatic hydrocarbon monovalent radical. This term is exemplified by groups such as methyl, ethyl, n-propyl, 1-methylethyl (isopropyl), n-butyl, n-pentyl, 1,1-dimethylethyl (tert-butyl), and the like. It may be abbreviated “Alk”.

The terms “alkenyl” or “alkenyl group” mean a branched or straight-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

The terms “alkynyl” or “alkynyl group” mean a branched or straight-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

The terms “alkylene” or “alkylene group” mean a branched or straight-chain saturated aliphatic hydrocarbon divalent radical having the specified number of carbon atoms. This term is exemplified by groups such as methylene, ethylene, propylene, n-butylene, and the like, and may alternatively and equivalently be denoted herein as -(alkyl)-.

The terms “alkenylene” or “alkenylene group” mean a branched or straight-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as -(alkylenyl)-.

The terms “alkynylene” or “alkynylene group” mean a branched or straight-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptenylene, octynylene, decynylene, and the like, and may alternatively and equivalently be denoted herein as -(alkynyl)-.

The terms “alkoxy” or “alkoxy group” mean a monovalent radical of the formula AlkO-, where Alk is an alkyl group. This term is exemplified by groups such as methoxy, ethoxy, propoxy, isopropoxy, butoxy, sec-butoxy, tert-butoxy, pentoxy, and the like.

The terms “alkoxycarbonyl” or “alkoxycarbonyl group” mean a monovalent radical of the formula AlkO-C(O)—, where Alk is alkyl. Exemplary alkoxycarbonyl groups include methoxycarbonyl, ethoxycarbonyl, tert-butyloxycarbonyl, and the like.

The term “alkoxycarbonylamino” or “alkoxycarbonylamino group” mean a monovalent radical of the formula ROC(O)NH—, where R is lower alkyl.

The terms “alkylcarbonylamino” or “alkylcarbonylamino group” or “alkanoylamino” or “alkanoylamino groups” mean a monovalent radical of the formula AlkC(O)NH—, where Alk is alkyl. Exemplary alkylcarbonylamino groups include acetamido (CH₃C(O)NH—).

The terms “alkylaminocarbonyloxy” or “alkylaminocarbonyloxy group” mean a monovalent radical of the formula AlkNHC(O)O—, where Alk is alkyl.

The terms “amino” or “amino group” mean an —NH₂ group.

The terms “alkylamino” or “alkylamino group” mean a monovalent radical of the formula (Alk)NH—, where Alk is alkyl. Exemplary alkylamino groups include methylamino, ethylamino, propylamino, butylamino, tert-butylamino, and the like.

The terms “dialkylamino” or “dialkylamino group” mean a monovalent radical of the formula (Alk)(Alk)N—, where each Alk is independently alkyl. Exemplary dialkylamino groups include dimethylamino, methylethylamino, diethylamino, dipropylamino, ethylpropylamino, and the like.

The terms “aminocarbonyl”, “alkylaminocarbonyl” or “dialkylaminocarbonyl” mean a monovalent radical of the formula R₂NC(O)—, where the R is independently hydrogen or alkyl.

The terms “substituted amino” or “substituted amino group” mean a monovalent radical of the formula —NR₂, where each R is independently a substituent selected from hydrogen or the specified substituents (but where both R_{S} cannot be hydrogen). Exemplary substituents include alkyl, alkanoyl, aryl, arylalkyl, cycloalkyl, heterocyclyl, heteroaryl, heteroarylalkyl, and the like.

The terms “alkoxycarbonylamino” or “alkoxycarbonylamino group” mean a monovalent radical of the formula AlkOC(O)NH—, where Alk is alkyl.

The terms “halogen” or “halogen group” mean a fluoro, chloro, bromo, or iodo group.

The term “halo” means one or more hydrogen atoms of the group are replaced by halogen groups.

The terms “alkylthio” or “alkylthio group” mean a monovalent radical of the formula AlkS-, where Alk is alkyl. Exemplary groups include methylthio, ethylthio, n-propylthio, isopropylthio, n-butylthio, and the like.

The terms “sulfonyl” or “sulfonyl group” mean a divalent radical of the formula —SO₂—.

The terms “aminosulfonyl”, “alkylaminosulfonyl” and “dialkylaminosulfonyl” mean a monovalent radical of the formula R₂N—SO₂—, wherein R is independently hydrogen or alkyl

The terms “aryl” or “aryl group” mean an aromatic carbocyclic monovalent or divalent radical of from 6 to 14 carbon atoms having a single ring (e.g., phenyl or phenylene) or multiple condensed rings (e.g., naphthyl or anthranyl). Unless otherwise specified, the aryl ring may be attached at any suitable carbon atom which results in a stable structure and, if substituted, may be substituted at any suitable carbon atom which results in a stable structure. Exemplary aryl groups include phenyl, naphthyl, anthryl, phenanthryl, indanyl, indenyl, biphenyl, and the like. It may be abbreviated “Ar”.

The term “compounds of the invention” and equivalent expressions are meant to embrace compounds of Formula (I) as herein described, including the tautomers, the prodrugs, the salts, particularly the pharmaceutically acceptable salts, and the solvates and hydrates thereof, where the context so permits. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

The terms “stable compound” or “stable structure” mean a compound that is sufficiently robust to survive isolation to a useful degree of purity from a reaction mixture, and formulation into an efficacious therapeutic or diagnostic agent. For example, a compound which would have a “dangling valency” or is a carbanion is not a compound contemplated by the invention.

The term “substituted” means that any one or more hydrogens on an atom of a group or moiety, whether specifically designated or not, is replaced with a selection from the indicated group of substituents, provided that the atom's normal valency is not exceeded and that the substitution results in a stable compound. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. For example, when the substituent is piperazinyl, piperidinyl, or tetrazolyl, unless specified otherwise, such piperazinyl, piperidinyl, or tetrazolyl group may be bonded to the rest of the compound of the invention via any atom in such piperazinyl, piperidinyl, or tetrazolyl group. Generally, when any substituent or group occurs more than one time in any constituent or compound, its definition on each occurrence is independent of its definition at every other occurrence. Such combinations of substituents and/or variables, however, are permissible only if such combinations result in stable compounds.

In a specific embodiment, the term “about” or “approximately” means within 20%, preferably within 10%, and more preferably within 5% of a given value or range.

The yield of each of the reactions described herein is expressed as a percentage of the theoretical yield.

### EXPERIMENTAL EXAMPLES

The invention provides processes for making compounds of Formula (X). In all schemes, unless specified otherwise, R¹ to R³ in the formulas below have the meanings of R¹ to R³ in the Summary of the Invention section. Intermediates used in the preparation of compounds of the invention are either commercially available or readily prepared by methods known to those skilled in the art.

The synthesis of a compound of Formula (X) is carried out as shown in Scheme I below.

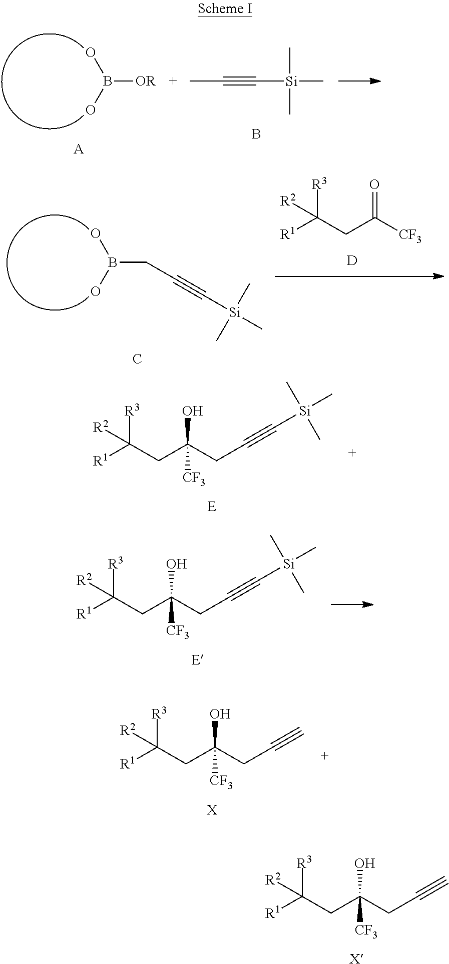

As illustrated in Scheme I, reacting a dioxaborolane of Formula (A) with a trimethylsilyl alkyne of Formula (B), in a suitable solvent, in the presence of a suitable base and with or without a metal halide, such as and preferably with magnesium chloride, provides an alkynyl borolane of Formula (C). Reacting the alkynyl borolane of Formula (C) with a suitable trifluoromethyl ketone of Formula (D), in the presence of a suitable organometallic reagent formed by the reaction of dialkyl zinc and N-alkyl-L-proline, in a suitable solvent, at a suitable temperature, and subsequently adding a suitable acid, such as phosphoric acid, to the reaction mixture, forms a mixture of trimethylsilyl alkynes of Formula (E) and (E′). Reacting the trimethylsilyl alkyne of Formula (E) or (E′) with a suitable base, such as sodium methoxide in a suitable solvent, at a suitable temperature, provides a compound of Formula (X) or (X′) respectively.

The compound of Formula (X) may be converted to another compound of Formula (X) by reactions known to one skilled in the art.

Optimum reaction conditions and reaction times may vary depending on the particular reactants used. Unless otherwise specified, solvents, temperatures, pressures, and other reaction conditions may be readily selected by one of ordinary skill in the art. Furthermore, if the substituent groups on R¹ to R³ are incompatible under the reaction conditions of the process, protection/deprotection of these groups may be carried out, as required, using reagents and conditions readily selected by one of ordinary skill in the art, see, for example, T. W. Greene and P. G. M. Wuts, Protective Groups in Organic Synthesis, New York: John Wiley & Sons (1999) and references cited therein. For example, a hydroxyl group can be protected as methyl ether and be deprotected at an appropriate stage with reagents, such as boron tribromide in dichloromethane. Specific procedures are provided in the Experimental Examples section. Typically, reaction progress may be monitored by high performance liquid chromatography (HPLC) or thin layer chromatography (TLC), if desired, and intermediates and products may be purified by chromatography on silica gel by recrystallization and/or distillation.

### Synthetic Examples

The following are representative examples that illustrate the process of the invention. HPLC used to determine diastereoselectivity were done on a Supelco SUPELCOSIL™ ABZ+Plus column (4.6 mm×10 cm) eluting with a gradient of 5% acetonitrile/95% water/0.05% TFA to 100% acetonitrile/0.05% TFA over 15 minutes and then held at 100% acetonitrile/0.05% TFA for 5 minutes. References to concentration or evaporation of solutions refer to concentration on a rotary evaporator.

### Example 1

### 5-Fluoro-2-[(3R)-3-trifluoromethyl-3-hydroxy-1,1-dimethylhex-5-ynyl]-N-[(1S)-1-(4-methoxyphenyl)ethyl]benzamide

### 1. Preparation of 2-(3-Trimethylsilyl-2-propynyl)-4,4,5,5-tetramethyl-1,3,2-dioxaborolane

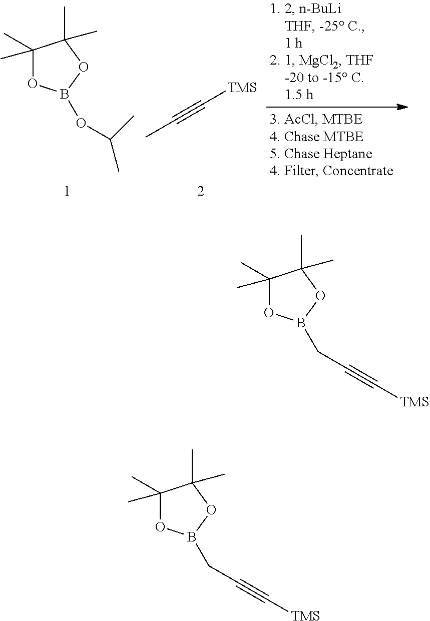

### 2-(3-Trimethylsilyl-2-propynyl)-4,4,5,5-tetramethyl-1,3,2-dioxaborolane

n-Butyl lithium (2.5 M in hexanes, 404 mL, 1.01 mol) was charged to an anhydrous solution of 1-trimethylsilylpropyne (118 g, 155.3 mL, 1.05 mol) in THF (<500 ppm water, 733 mL) at a rate such that the temperature was maintained between −20° C. and −25° C. After aging the solution for 1 hour at −20° C. to −25° C., the solution while at −20° C. to −25° C. was charged to a suspension of magnesium chloride (anhydrous, 93.3 g, 0.98 mol) and 2-isopropoxy-4,4,5,5-tetramethyl-1,3,2-dioxaborolane (182 g, 200 mL, 0.98 mol) in THF at a rate such that the reaction temperature was maintained at −20° C. to −25° C. The mixture was agitated at −20° C. to −25° C. for 2 hours; at which point, a solution of acetyl chloride (80 g, 72 mL, 1.10 mol) in MTBE (72 mL) was charged to the reaction mixture such that the reaction temperature did not exceed −20° C. The reaction mixture was aged at −20° C. and −25° C. for 1 hour, at which point the reaction was warmed to 20° C. The reaction mixture was concentrated in vacuo to approximately one-third (⅓) of the original volume and chased with one 1 L portion and then one 700 mL portion of tert-butyl methyl ether (MTBE) and finally one 1 L portion of heptane. The mixture was diluted with 350 mL of heptane, filtered, and the solids were rinsed with heptane. The filtrated was concentrated in vacuo to afford 2-(3-trimethylsilyl-2-propynyl)-4,4,5,5-tetramethyl-1,3,2-dioxaborolane as an orange oil (225.10 g, 80.8 wt. % by assay NMR, 77.9% yield). The oil can be further purified by distillation to afford 2-(3-trimethylsilyl-2-propynyl)-4,4,5,5-tetramethyl-1,3,2-dioxaborolane in 97 wt. %.

### 2. Preparation of 5-Fluoro-2-[(3R)-3-trifluoromethyl-3-hydroxy-1,1-dimethyl-6-trimethylsilylhex-5-ynyl]-N-[(1S)-1-(4-methoxyphenyl)ethyl]benzamide and 5-Fluoro-2-[(3S)-3-trifluoromethyl-3-hydroxy-1,1-dimethyl-6-trimethylsilylhex-5-ynyl]-N-[(1S)-1-(4-methoxyphenyl)ethyl]benzamide

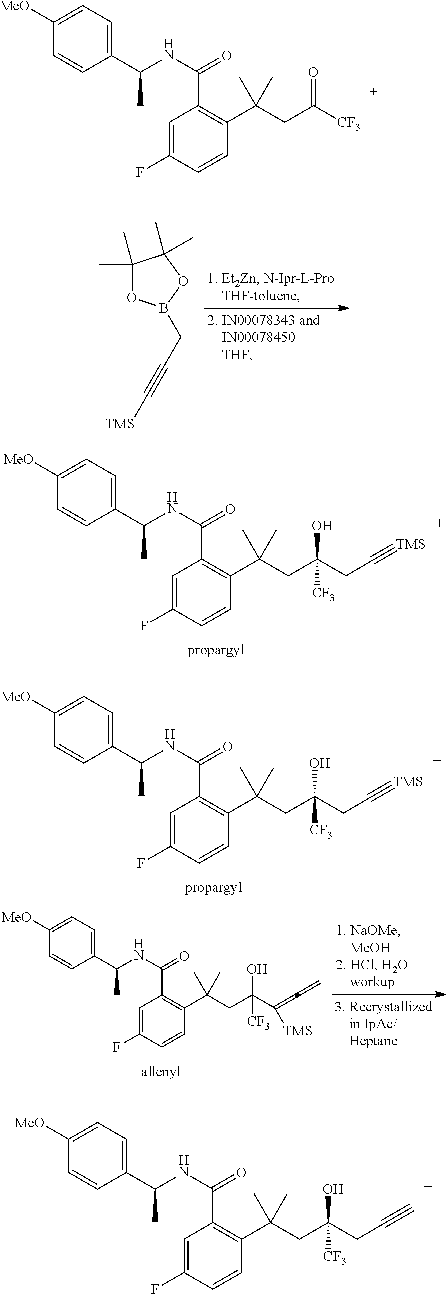 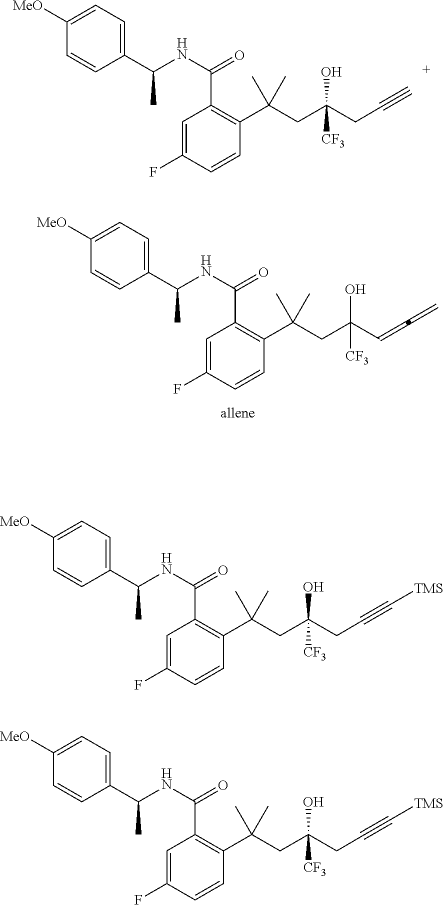 [0069] 5-Fluoro-2-[(3R)-3-trifluoromethyl-3-hydroxy-1,1-dimethyl-6-trimethylsilylhex-5-ynyl]-N—[(1S)-1-(4-methoxyphenyl)ethyl]benzamide
5-Fluoro-2-[(3S)-3-trifluoromethyl-3-hydroxy-1,1-dimethyl-6-trimethylsilylhex-5-ynyl]-N—[(1S)-1-(4-methoxyphenyl)ethyl]benzamide

### −20° C. Procedure for High Diastereoselectivity

Diethyl zinc (1.1 M solution in toluene, 1.28 mL, 1.41 mmol) was charged to a suspension of N-isopropyl-L-proline (222 mg, 1.41 mmol) in THF (<50 ppm water, 7 mL) under nitrogen such that the reaction temperature did not exceed 25° C. The reaction mixture was warmed to 35° C. and agitated at this temperature for 1.5 hours to afford a homogenous solution. The solution was cooled to −20° C., at which point 2-(3-trimethylsilyl-2-propynyl)-4,4,5,5-tetramethyl-1,3,2-dioxaborolane (373 mg, 90.0 wt %, 1.410 mmol) followed by a solution of 5-fluoro-N—[(S)-1-(4-methoxyphenyl)ethyl]-2-(4,4,4-trifluoro-1,1-dimethyl-3-oxobutyl)benzamide (300 mg, 0.705 mmol) in THF (<50 ppm water, 1 mL) were charged to the above solution at −20° C. The reaction was aged for 3 days at −20° C., at which point HPLC (220 nm) analysis showed >93% molar conversion. The reaction was quenched with phosphoric acid (0.15 M, 5 mL) and diluted with acetonitrile (100 mL). The solution was analyzed by HPLC (220 nm) to show 7.9:1 dr favoring 5-fluoro-2-[(3R)-3-trifluoromethyl-3-hydroxy-1,1-dimethyl-6-trimethylsilylhex-5-ynyl]-N-[(1S)-1-(4-methoxyphenyl)ethyl]benzamide over 5-fluoro-2-[(3S)-3-trifluoromethyl-3-hydroxy-1,1-dimethyl-6-trimethylsilylhex-5-ynyl]-N-[(1S)-1-(4-methoxyphenyl)ethyl]benzamide HPLC (220 nm) analysis also showed 8:1 propargyl to allenyl products and an 76% assay yield for 5-fluoro-2-[(3R)-3-trifluoromethyl-3-hydroxy-1,1-dimethylhex-5-ynyl]-N-[(1S)-1-(4-methoxyphenyl)ethyl]benzamide.

### 3. Preparation of 5-Fluoro-2-[(3R)-3-trifluoromethyl-3-hydroxy-1,1-dimethylhex-5-ynyl]-N-[(1S)-1-(4-methoxyphenyl)ethyl]-benzamide

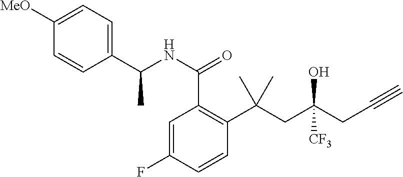

### 5-Fluoro-2-[(3R)-3-trifluoromethyl-3-hydroxy-1,1-dimethylhex-5-ynyl]-N-[(1S)-1-(4-methoxyphenyl)ethyl]benzamide

### 20° C. Procedure for High Conversion and Isolation

Diethyl zinc (2.3 M solution in toluene, 103 mL, 0.235 mol) was charged to a suspension of N-isopropyl-L-proline (38.3 g, 0.241 mol) in THF (<50 ppm water, 412 mL) under nitrogen at a rate such that the temperature did not exceed 25° C. The reaction mixture warmed to 40° C. and aged at this temperature for 3 hours to afford a homogeneous solution. The solution was cooled to 20° C., at which point a solution of 5-fluoro-N—[(S)-1-(4-methoxyphenyl)ethyl]-2-(4,4,4-trifluoro-1,1-dimethyl-3-oxobutyl)benzamide (50.0 g, 118 mmol) and 2-(3-trimethylsilyl-2-propynyl)-4,4,5,5-tetramethyl-1,3,2-dioxaborolane (62.6 g, 89.5 wt. %, 0.235 mol) in THF (<50 ppm water, 100 mL) was charged to the reaction dropwise over 5 hours. The reaction mixture was aged for 10 hours, at which point HPLC analysis showed 3.5:1 dr favoring 5-fluoro-2-[(3R)-3-trifluoromethyl-3-hydroxy-1,1-dimethyl-6-trimethylsilylhex-5-ynyl]-N-[(1S)-1-(4-methoxyphenyl)ethyl]benzamide over 5-fluoro-2-[(3S)-3-trifluoromethyl-3-hydroxy-1,1-dimethyl-6-trimethylsilylhex-5-ynyl]-N—[(1S)-1-(4-methoxyphenyl)ethyl]benzamide and 25:1 propargyl to allenyl products.

The reaction was carefully quenched with 230 mL of aqueous HCl (3 M) at a rate such that the temperature did not exceed 25° C. and to control the gas (ethane) evolution. The layers were separated and the organic portion was washed with 100 mL of water. Sodium methoxide (25 wt. % in methanol, 65.0 mL, 284 mmol) was charged to the reaction, and the reaction was aged for 1 hour at 30° C. The reaction was cooled to 20° C., quenched by the addition of 83 mL of aqueous HCl (3M) and diluted with 150 mL of water. After stirring the mixture for 10 minutes, the pH of the aqueous phase was adjusted to 6.0 by the addition of 10 mL of aqueous HCl (3M), the organic layer was removed by distillation, and the mixture was diluted with 400 mL of isopropyl acetate. The mixture was stirred for 20 minutes and charged with 10 mL of aqueous HCl (3M). The layers were separated, and the organic portion was washed with 100 mL of water. The organic layer was concentrated to a solid and the mixture was recrystallized with isopropyl acetate and heptane to afford 5-fluoro-2-[(3R)-3-trifluoromethyl-3-hydroxy-1,1-dimethylhex-5-ynyl]-N-[(1S)-1-(4-methoxyphenyl)ethyl]benzamide as a tan powder (38.5 g, 98.2 wt. %, 69% yield).

What is claimed is:

1. A process for synthesis of a compound of Formula (X) 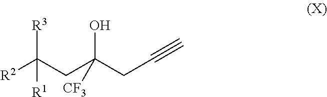
wherein:
R¹ is an aryl group substituted with one to three substituent groups,
wherein each substituent group of R¹ is independently C₁-C₅ alkyl, aminocarbonyl, alkylaminocarbonyl, dialkylaminocarbonyl, halogen, carboxy, cyano, or trifluoromethyl,
wherein each substituent group of R¹ is optionally independently substituted with one to three substituents selected from C₁-C₃ alkyl, C₁-C₃ alkoxy, phenyl, and alkoxyphenyl; and
R² and R³ are each independently C₁-C₅ alkyl;
the process comprising:
(a) reacting a dioxaborolane of Formula (A) with a trialkylsilyl alkyne of Formula (B), in a suitable solvent, in the presence of a suitable base with or without a metal halide, such as magnesium chloride, and subsequently adding acetyl chloride to provide an alkynyl borolane of Formula (C) 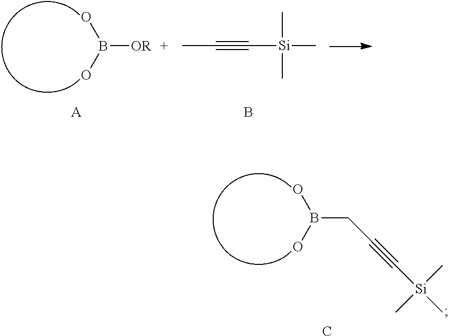
(b) reacting the alkynyl borolane of Formula (C) with a suitable trifluoromethyl ketone of Formula (D), in the presence of a organometallic complex generated from the reaction of dialkyl zinc and a suitable N-alkyl-L-proline, in a suitable solvent, at a suitable temperature, and subsequently adding a suitable acid, such as phosphoric acid, to the reaction mixture to form a mixture of trimethylsilyl alkynes of Formula (E) and (E′) 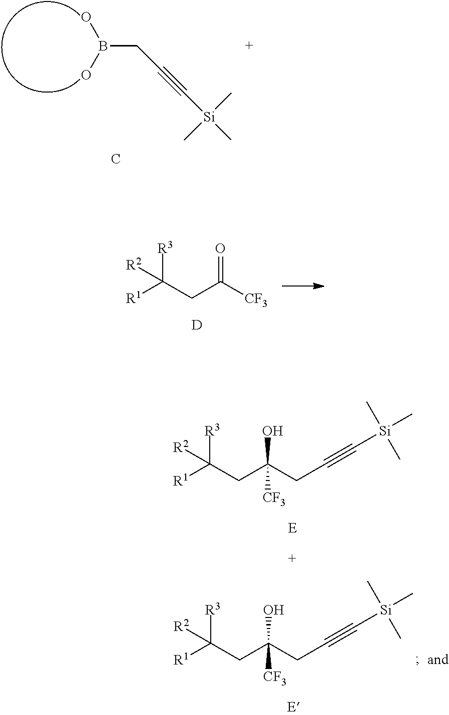
(c) reacting the trimethylsilyl alkyne of Formula (E) or (E′) with a suitable base, such as sodium hydroxide or an alkoxide base, at a suitable temperature, to provide a compound of Formula (X) or (X′) respectively

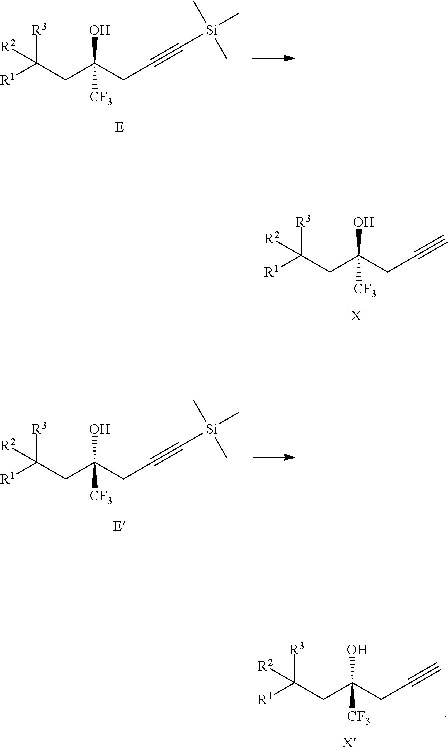

2. The process according to claim 1, wherein:
R¹ is an aryl group substituted with one to three substituent groups,
wherein each substituent group of R¹ is independently C₁-C₅ alkyl, aminocarbonyl, alkylaminocarbonyl, halogen, carboxy, cyano, or trifluoromethyl,
wherein each substituent group of R¹ is optionally independently substituted with one to three substituents selected from C₁-C₃ alkyl, phenyl, and alkoxyphenyl; and
R² and R³ are each independently C₁-C₃ alkyl.

3. The process according to claim 1, wherein the dioxaborolane of step (a) is 2-methoxy-4,4,5,5-tetramethyl-1,3,2-dioxaborolane or 2-isopropoxy-4,4,5,5-tetramethyl-1,3,2-dioxaborolane.

4. The process according to claim 1, wherein the trialkylsilane alkyne of step (a) is 1-triethylsilyl-1-propyne, 1-trimethylsilyl-1-propyne, 1-triisopropylsilyl-1-propyne, 1-(t-butyl-dimethylsilyl)-1-propyne, or 1-(tert-butyldiphenylsilyl)-1-propyne.

5. The process according to claim 4, wherein the trialkylsilane alkyne of step (a) is 1-trimethylsilyl-1-propyne.

6. The process according to claim 1, wherein the suitable solvent of step (a) is diethyl ether, dipropyl ether, diisopropyl ether, dibutyl ether, tetrahydrofuran (THF), ethylene glycol dimethyl ether (DME), tert-butyl methyl ether (MTBE), or a mixture thereof.

7. The process according to claim 6, wherein the suitable solvent of step (a) is diethyl ether or THF.

8. The process according to claim 1, wherein the suitable base for step (a) is n-butyl lithium, sec-butyl lithium, tert-butyl lithium, or n-pentyl lithium.

9. The process according to claim 8, wherein the suitable base for step (a) is n-butyl lithium.

10. The process according to claim 1, wherein the suitable metal halide for step (a) is magnesium chloride, magnesium bromide, or magnesium triflate.

11. The process according to claim 10, wherein the suitable metal halide for step (a) is magnesium chloride.

12. The process according to claim 1, wherein the trifluoromethyl ketone compound (D) for step (b) is 5-fluoro-N-(4-methoxybenzyl)-2-(4,4,4-trifluoro-1,1-dimethyl-3-oxobutyl)benzamide, 4-(5-bromo-2-methoxyphenyl)-1,1,1-trifluoro-4-methylpentan-2-one, or 5-fluoro-N—[(S)-1-(4-methoxyphenyl)ethyl]-2-(4,4,4-trifluoro-1,1-dimethyl-3-oxobutyl)benzamide.

13. The process according to claim 1, wherein the suitable aqueous acid of step (b) is hydrochloride acid, hydrobromic acid, sulfuric acid, trifluoroacetic acid, acetic acid, phosphoric acid, or ammonium chloride.

14. The process according to claim 13, wherein the suitable aqueous acid of step (b) is aqueous hydrochloric acid.

15. The process according to claim 1, wherein the suitable dialkyl zinc of step (b) is dimethyl zinc, diethyl zinc, or diisopropyl zinc.

16. The process according to claim 15, wherein the suitable dialkyl zinc of step (b) is diethyl zinc.

17. The process according to claim 1, wherein the suitable N-alkyl-L-proline of step (b) is N-methyl-L-proline, N-ethyl-L-proline, N-isobutyl-L-proline, N-isopropyl-L-proline, N-cyclopentyl-L-proline, N-cyclohexyl-L-proline, N-tert-butyl-L-proline, or N-3-pentyl-L-proline.

18. The process according to claim 18, wherein the suitable N-alkyl-L-proline of step (b) is N-isopropyl-L-proline or N-cyclopentyl-L-proline.

19. The process according to claim 1, wherein the suitable temperature of step (b) is from −78° C. to 30° C.

20. The process according to claim 1, wherein the suitable base of step (c) is sodium hydroxide, potassium hydroxide, cesium hydroxide, sodium methoxide, sodium ethoxide, sodium isopropoxide, or sodium tert-butoxide.

21. The process according to claim 20, wherein the suitable base of step (c) is sodium methoxide.

22. The process according to claim 1, wherein the suitable temperature of step (c) is 0° C. to 50° C.
